(19) United States
(12) Patent Application Publication
(10) Pub. No.: US 2020/0030210 A1
(43) Pub. Date: Jan. 30, 2020

(54) IMPROVED CONDITIONING HAIR TREATMENT PRODUCT WITH WASHOUT PROTECTION

(71) Applicant: Henkel AG & Co. KGaA, Duesseldorf (DE)

(72) Inventors: VOLKER SCHEUNEMANN, Lueneburg (DE); ERIK SCHULZE ZUR WIESCHE, Hamburg (DE); RENE KROHN, Norderstedt (DE)

(73) Assignee: Henkel AG & Co. KGaA, Duesseldorf (DE)

(21) Appl. No.: 16/337,306

(22) PCT Filed: Jul. 24, 2017
(86) PCT No.: PCT/EP2017/068603

§ 371 Date: Mar. 27, 2019

(30) Foreign Application Priority Data
Sep. 30, 2016 (DE) 10 2016 219 003.2

(51) Int. Cl. A61K 8/49; A61K 8/65; A61K 8/73; A61K 8/41; A61K 8/34; A61K 8/44; A61Q 5/12; A61Q 5/02

(52) U.S. Cl. CPC A61K 8/4913; A61K 8/65; A61K 8/731; A61K 8/737; A61Q 5/02; A61K 8/34; A61K 8/345; A61K 8/44; A61Q 5/12; A61K 8/416

(57) ABSTRACT

Hair treatment agents and methods for treating hair are presented herein. An exemplary hair treatment agent includes, based on its weight, (a) from about 0.001 to about 10 wt. % of at least one succinimidyl ester, and (b) from about 0.001 to about 20 wt. % of at least one proteolipid of formula (II) R′—X—R″. In formula (II), R′ represents a straight-chain or branched, saturated or unsaturated hydrocarbon functional group having 11 to 24 carbon atoms; R″ represents a protein, a peptide or a protein hydrolysate; and X represents —C(O)O— or —N⁺(R^{III}₂)—R^{IV}— or —N(R^{III})R^{IV}— or —C(O)—N(R^{V})R^{VI}, where R^{III} represents —(CH₂)ₓ—CH₃, where x=0-22, and R^{IV} represents —CH₂—CH(OH)—CH₂— or —(CH₂)_{X}—, where x=0-22; and R^{V} and R^{VI} represent, independently of one another, —H or —(CH₂)_{X}—CH₃, where x=0-22; wherein R″ represents keratin or a keratin hydrolyzate when X represents —C(O)O—.

### CROSS-REFERENCE TO RELATED APPLICATION

This application is a U.S. National-Stage entry under 35 U.S.C. § 371 based on International Application No. PCT/EP2017/068603, filed Jul. 24, 2017, which was published under PCT Article 21(2) and which claims priority to German Application No. 10 2016 219 003.2, filed Sep. 30, 2016, which are all hereby incorporated in their entirety by reference.

### TECHNICAL FIELD

The present disclosure relates to hair treatment agents, in particular shampoos and what are referred to as conditioners, having an active ingredient combination for the gentle and effective care of hair.

### BACKGROUND

The importance of care products with the longest-lasting possible effect is increasing not least due to the high amount of strain on hair caused, for example, by coloring or permanents, by cleaning hair with shampoos, and by environmental factors. Care products of this type influence the natural structure and properties of hair. For example, following care treatments of this kind, the wet and dry combability, the hold and the volume of hair can be optimized, or hair can be protected from increased splitting.

It has therefore long been customary to subject hair to a special aftertreatment. In this process, the hair is treated with special active ingredients, for example quaternary ammonium salts or special polymers, usually in the form of a rinse. As a result of this treatment, depending on the formulation, the combability, the hold, and the volume of the hair are improved, and the amount of splitting is reduced.

Multi-functional cosmetic products are also known in the prior art. These include in particular what are referred to as “2 in 1” shampoos, which not only clean hair but also condition it. Products of this type are held in high esteem by consumers because, due to their product performance, they eradicate the need for at least one process step, for example conditioning with a conventional hair conditioner.

Similarly, products for modifying natural hair color play a prominent role in hair cosmetics. A distinction is made between permanent, semi-permanent, or temporary coloring systems based on chemical and/or natural dyes. Hair colors produced artificially by permanent, semi-permanent or temporary coloring systems are, however, disadvantageous in that they can change undesirably, for example during or after hair cleaning.

An “undesirable change” is understood to mean fading or bleeding and loss of the color brilliance of the hair shade achieved by the particular coloring. Environmental impacts and/or the effects of sunlight can further intensify these changes.

There continues to be a need to provide active ingredients or active ingredient combinations for hair treatment agents which have good nourishing properties and also strengthen the adhesion of dyes to the hair fibers and thus maintain the fastness of the artificially produced hair color, and to develop hair treatment agents in this respect.

It has been observed, however, that water hardness can sometimes negatively influence the nourishing properties of hair treatment agents, and therefore the nourishing performance of the same product may be either too low or too high depending on water quality.

Nourishing performance that is too high or “over-nourishment” (occurrence of what is known as a build-up effect) of the hair is understood to mean in particular a greasy hair feel, lack of hair volume, and/or a lank, unkempt appearance of the hair, whereas damaged hair (broken and dull hair that is prone to splitting) is an indicator of nourishing performance that is too low.

The object of the present application was therefore to provide skin-compatible, nourishing hair treatment agents which have excellent foaming properties, can be rinsed out well with water, and additionally reduce or prevent the washout of color from colored hair.

The nourishing hair treatment agents should have a uniform conditioning performance irrespective of water quality and in particular should not weigh down fine hair and/or damaged hair, but should strengthen the structure of the hair and thus protect it from splitting and breaking and should improve the combability and feel of the hair. In addition, said agents should have a germicidal effect.

### BRIEF SUMMARY

Hair treatment agents and methods for treating hair are presented herein. An exemplary hair treatment agent includes, based on its weight, (a) from about 0.001 to about 10 wt. % of at least one succinimidyl ester, and (b) from about 0.001 to about 20 wt. % of at least one proteolipid of formula (II) R′—X—R″. In formula (II), R′ represents a straight-chain or branched, saturated or unsaturated hydrocarbon functional group having 11 to 24 carbon atoms; R″ represents a protein, a peptide or a protein hydrolysate; and X represents —C(O)O— or —N⁺(R^{III}₂)—R^{IV}— or —N(R^{III})R^{IV}— or —C(O)—N(R^{V})R^{VI}, where R^{III} represents —(CH₂)ₓ—CH₃, where x=0-22, and R^{V} represents —CH₂—CH(OH)—CH₂— or —(CH₂)_{X}—, where x=0-22; and R^{V} and R^{VI} represent, independently of one another, —H or —(CH₂)_{X}—CH₃, where x=0-22; wherein R^{IV} represents keratin or a keratin hydrolyzate when X represents —C(O)O—.

In another embodiment, a method for treating hair includes applying to wet or dry hair a hair treatment agent comprising, based on its weight, (a) from about 0.001 to about 10 wt. % of at least one succinimidyl ester, and (b) from about 0.001 to about 20 wt. % of at least one proteolipid of formula (II) R′—X—R″. In formula (II), R′ represents a straight-chain or branched, saturated or unsaturated hydrocarbon functional group having 11 to 24 carbon atoms; R″ represents a protein, a peptide or a protein hydrolysate; and X represents —C(O)O— or —N⁺(R^{III}₂)—R^{IV}— or —N(R^{III})R^{IV}— or —C(O)—N(R^{V})R^{VI}, where R^{III} represents —(CH₂)ₓ—CH₃, where x=0-22, and R^{V} represents —CH₂—CH(OH)—CH₂— or —(CH₂)_{X}—, where x=0-22; and R^{V} and R^{VI} represent, independently of one another, —H or —(CH₂)_{X}—CH₃, where x=0-22; wherein R″ represents keratin or a keratin hydrolyzate when X represents —C(O)O—. The method further includes leaving the hair treatment agent on the hair for a selected period of time, and subsequently rinsing out the hair treatment agent.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has now been found that a combination of certain ingredients has a particularly positive effect on colored hair and the hair follicles treated with said ingredients.

The present disclosure relates firstly to hair treatment agents containing, based on their weight,
a) from about 0.001 to about 10 wt. % of at least one succinimidyl ester,
b) from about 0.001 to about 20 wt. % of at least one proteolipid of formula (II)

R′—X—R″ (II).
in which
   R′ represents a straight-chain or branched, saturated or unsaturated hydrocarbon functional group having 11 to 24 carbon atoms,
   R″ represents a protein, a peptide or a protein hydrolyzate,
   X represents —C(O)O— or —N⁺(R^{II}₂)—R^{IV}— or —N(R^{III})R^{IV}— or —C(O)—N(R^{V})R^{VI},
   R^{III} represents —(CH₂)ₓ—CH₃, where x=0-22, and
R^{IV} represents —CH₂—CH(OH)—CH₂— or —(CH₂)_{X}—, where x=0-22;
R^{V} and R^{VI} represent, independently of one another, —H or —(CH₂)_{X}—CH₃, where x=0-22;
with the proviso that R″ represents keratin or a keratin hydrolyzate when X represents —C(O)O—.

Hair treatment agents within the meaning of the present disclosure are, for example, hair shampoos, hair conditioners, conditioning shampoos, hair sprays, hair rinses, hair masks, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, agents that men use already are preferred. Preferred agents are therefore shampoos, conditioning agents, or hair tonics.

The hair treatment agents contain, as a first essential ingredient, from about 0.001 to about 10 wt. % of at least one succinimidyl ester.

Succinimidyl esters are esters of carboxylic acids having (optionally substituted) N-hydroxysuccinimide (NHS, IUPAC 1-hydroxy-2,5-pyrrolidinedione) and are also referred to as NHS esters. Hair treatment agents which are preferred as contemplated herein additionally contain, based on the weight of the agent, from about 0.01 to about 10 wt. %, preferably from about 0.05 to about 7.5 wt. %, more preferably from about 0.1 to about 6 wt. %, and in particular from about 0.15 to about 5 wt. % succinimidyl ester(s) of formula (III)

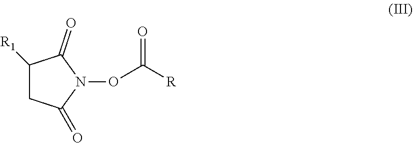

in which
R1 represents —H or an ionic group.
R represents an optionally substituted saturated or unsaturated, linear, branched or cyclic, aliphatic or aromatic hydrocarbon functional group having at least 5 C atoms.

Suitable ionic groups R1 include both anionic groups such as phosphate, phosphonate, phosphinate, sulfate, sulfonate, sulfinate, sulfenate, oxysulfonate, and carboxylate groups, as well as cationic groups such as substituted or unsubstituted ammonium groups. Zwitterionic/betaine groups such as carboxybetaine or sulfobetaine groups are also possible.

In succinimidyl esters which are preferred as contemplated herein, R1 represents —H, —OSO₃⁻, —SO₃⁻, —SO₂⁻, —COO⁻, —NH₃⁺, —N(CH₃)H₂⁺, —N(CH₃)₂H⁺, —N(CH₃)₃⁺, —N⁺(CH₃)₂(CH₂)₂—COO⁻, or —N⁺(CH₃)₂(CH₂)₃—SO₃⁻.

Succinimidyl esters that are preferably to be used as contemplated herein are described on pages 4 to 7 of the priority document as numbers 1 to 72.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. % of at least one succinimidyl ester from the group

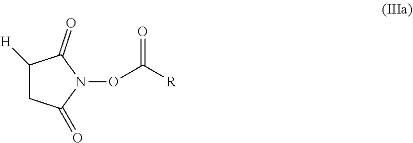

in which R represents -Ph or —(CH₂)nCH₃, where n=4, 5, 6, 7, 8, 9 or 10,

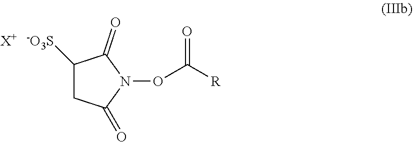

in which R represents -Ph or —(CH₂)nCH₃, where n=4, 5, 6, 7, 8, 9 or 10, and
X represents H, a monovalent cation, or the n-th part of an n-valent cation.

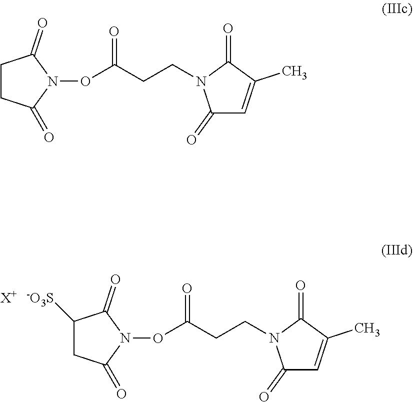

Hair treatment agents containing these succinimidyl esters have excellent nourishing properties on both thin and normal hair, bring about significant structure strengthening, and prevent washout and bleeding of chemically colored hair very effectively.

As further essential components, the agents as contemplated herein contain from about 0.001 to about 20 wt. % of at least one proteolipid of formula (I)

R′—X—R″ (I),

in which
R′ represents a straight-chain or branched, saturated or unsaturated hydrocarbon functional group having 11 to 24 carbon atoms,
R″ represents a protein, a peptide or a protein hydrolyzate,
X represents —C(O)O— or —N⁺(R^{III}₂)—R^{IV}— or —N(R^{II})R^{IV}— or —C(O)—N(R^{V})R^{VI}—,
R^{III} represents —(CH₂)ₓ—CH₃, where x=0-22, and
R^{IV} represents —CH₂—CH(OH)—CH₂— or —(CH₂)_{X}—, where x=0-22;
R^{V} and R^{VI} represent, independently of one another, —H or —(CH₂)_{X}—CH₃, where x=0-22;
   with the proviso that R″ represents keratin or a keratin hydrolyzate when X represents —C(O)O—.

The proteolipids are preferably used within certain amounts in the agents. Preferred cosmetic agents contain, based on their weight, from about 0.01 to about 10 wt. %, preferably from about 0.02 to about 5 wt. %, particularly preferably from about 0.05 to about 2.5 wt. %, more preferably from about 0.1 to about 1 wt. % and in particular from about 0.15 to about 0.5 wt. %, of proteolipid(s).

The functional group R″ in formula (I) represents a peptide or a protein or a protein hydrolyzate. When X=—C(O)O—, R″ is selected from the group of keratin or keratin hydrolyzate.

Preferred functional groups R″ are oligopeptides which have at least one amino acid sequence Glu-Glu-Glu, it being possible for the amino group to be present in a free or protonated form and for the carboxy groups to be present in a free or deprotonated form.

Within the context of the present application, oligopeptides are condensation products of amino acids which are linked by peptide bonds in the manner of an acid amide and which comprise at least about 3 and at most about 25 amino acids.

In preferred cosmetic agents of the embodiment described above, the oligopeptide (=the functional group R″) comprises from about 5 to about 15 amino acids, preferably from about 6 to about 13 amino acids, particularly preferably from about 7 to about 12 amino acids, and in particular about 8, about 9 or about 10 amino acids.

Depending on whether further amino acids are bound to the sequence Glu-Glu-Glu and depending on the type of these amino acids and also depending on the choice of the functional groups R′ and optionally R^{m} and R″, the molar mass of the proteolipid contained in the agents may vary. Preferred cosmetic agents are exemplified in that the proteolipid has a molar mass of from about 1,000 to about 30,000 daltons, preferably from about 1,250 to about 25,000 daltons, particularly preferably from about 1,500 to about 20,000 daltons, and in particular from about 2,000 to about 15,000 daltons.

Oligopeptides which not only consist of the three glutamic acids but also have further amino acids bound to this sequence are preferably used as the functional group R″. These further amino acids are preferably selected from certain amino acids, whereas certain other representatives are less preferred.

It is preferred if the functional group R″ of the proteolipids used in the agents contains tyrosine. It is further preferred if the functional group R″ of the proteolipids used in the agents contains leucine.

It is further preferred if the functional group R″ of the proteolipids used in the agents contains isoleucine.

It is further preferred if the functional group R″ of the proteolipids used in the agents contains arginine.

It is further preferred if the functional group R″ of the proteolipids used in the agents contains valine. Amino acid sequences contained in particularly preferred oligopeptides or contained in the preferred oligopeptides as the functional group R″ are described below:

A particularly preferred oligopeptide additionally contains tyrosine, which is preferably bound via its acid function to the sequence Glu-Glu-Glu. Preferred cosmetic agents are therefore exemplified in that the oligopeptide contained in the proteolipids of formula (I) as the functional group R″ has at least one amino acid sequence Tyr-Glu-Glu-Glu, it being possible for the amino group to be present in a free or protonated form and for the carboxy groups to be present in a free or deprotonated form.

A further particularly preferred oligopeptide additionally contains isoleucine, which is preferably bound via its amino function to the sequence Glu-Glu-Glu. Preferred cosmetic agents are therefore exemplified in that the oligopeptide contained in the proteolipids of formula (I) as the functional group R″ has at least one amino acid sequence Glu-Glu-Glu-Ile, it being possible for the amino groups to be present in a free or protonated form and for the carboxy groups to be present in a free or deprotonated form.

Oligopeptides that have both of the aforementioned amino acids (tyrosine and isoleucine) are preferred. In this case, cosmetic agents are particularly preferred in which the oligopeptide contained in the proteolipids of formula (I) as the functional group R″ has at least one amino acid sequence Tyr-Glu-Glu-Glu-Ile, it being possible for the amino group to be present in a free or protonated form and for the carboxy groups to be present in a free or deprotonated form.

Further preferred oligopeptides additionally contain arginine, which is preferably present bound to isoleucine. Preferred cosmetic agents are therefore exemplified in that the oligopeptide contained in the proteolipids of formula (I) as the functional group R″ has at least one amino acid sequence Tyr-Glu-Glu-Glu-Ile-Arg, it being possible for the amino groups to be present in a free or protonated form and for the carboxy groups to be present in a free or deprotonated form.

Still further preferred oligopeptides additionally contain valine, which is preferably present bound to the arginine. Further preferred cosmetic agents are therefore exemplified in that the oligopeptide contained in the proteolipids of formula (I) as the functional group R″ has at least one amino acid sequence Tyr-Glu-Glu-Glu-Ile-Arg-Val, it being possible for the amino groups to be present in a free or protonated form and for the carboxy groups to be present in a free or deprotonated form.

Still further preferred oligopeptides additionally contain leucine, which is preferably present bound to valine. Further preferred cosmetic agents are exemplified in that the oligopeptide contained in the proteolipids of formula (I) as the functional group R″ has at least one amino acid sequence Tyr-Glu-Glu-Glu-Ile-Arg-Val-Leu, it being possible for the amino groups to be present in a free or protonated form and for the carboxy groups to be present in a free or deprotonated form.

Particularly preferred oligopeptides additionally contain leucine, which is preferably present bound to the tyrosine. Further preferred cosmetic agents are exemplified in that the oligopeptide contained in the proteolipids of formula (I) as the functional group R″ has at least one amino acid sequence Leu-Tyr-Glu-Glu-Glu-Ile-Arg-Val-Leu, it being possible for the amino groups to be present in a free or protonated form and for the carboxy groups to be present in a free or deprotonated form.

In summary, cosmetic agents are particularly preferred which contain at least one proteolipid of formula (I) in which R″ has at least one amino acid sequence Leu-Tyr-Glu-Glu-Glu-Ile-Arg-Val-Leu, it being possible for the amino groups to be present in a free or protonated form and for the carboxy groups to be present in a free or deprotonated form.

As already mentioned, R″ is selected from the group of keratin or keratin hydrolyzate when X=—C(O)O—.

In all other cases, the functional group R″ in formula (I) may represent a peptide or a protein or a protein hydrolyzate, protein hydrolyzates being preferred. Protein hydrolyzates are product mixtures that are obtained by acid-, base-, or enzyme-catalyzed degradation of proteins. Protein hydrolyzates of both plant and animal origin may be used.

Animal protein hydrolyzates are, for example, elastin, collagen, keratin, silk, and milk protein hydrolyzates, which can also be present in the form of salts. Such products are marketed, for example, under the trademarks Dehylan® (BASF SE), Promois® (Interorgana), Collapuron® (BASF SE), Nutrilan® (BASF SE), Gelita-Solo (Deutsche Gelatine Fabriken Stoess & Co), Lexein® (Inolex), and Kerasol® (Croda).

It is preferable to use protein hydrolyzates of plant origin, for example soybean, almond, rice, pea, potato and wheat protein hydrolyzates. Such products are available, for example, under the trademarks Gluadin® (BASF SE), DiaMin® (Diamalt), Lexein® (Inolex), and Crotein® (Croda).

The functional group R″ is preferably selected from keratin or keratin hydrolyzates, irrespective of the choice of X in formula (I). Preferred cosmetic agents contain at least one proteolipid of formula (I) in which R″ represents keratin or a keratin hydrolyzate.

Particularly preferred groups R—X— are listed in the tables of WO 2011/042323 A2. Reference is explicitly and entirely made hereto with regard to the content thereof. The groups disclosed therein are bound to keratin or keratin hydrolyzate as the group R″ in still further preferred embodiments of the present disclosure.

Irrespective of the choice of group —X— in formula (I), cosmetic agents are preferred which contain at least one proteolipid of formula (I) in which R^{III} represents —CH₃ and R^{IV} represents —(CH₂)ₓ—, where x=0, 1, 2, 3, 4, 5, 6, 7, or 8.

Furthermore, particularly preferred cosmetic agents contain at least one proteolipid of formula (I) in which X represents —N⁺(CH₃)₂—CH₂—CH(OH)—CH₂— and R′ represents —(CH₂)₁₇—CH₃.

Cosmetic agents that are also particularly preferred contain at least one proteolipid of formula (I) in which X represents —N⁺(CH₃)₂—CH₂—CH(OH)—CH₂— and R′ represents —(CH₂)₁₋₂₃—CH₃.

Cosmetic agents that are also further preferred contain at least one proteolipid of formula (I) in which X represents —C(O)—O— and R′ represents —(CH₂)₁₇—CH₃.

The agents contain the proteolipid(s) preferably in an amount of from about 0.01 to about 10.0 wt. % and particularly preferably from about 0.05 to about 2.0 wt. %, in each case based on the weight of the ready-to-apply agent.

In summary, preferred hair treatment agents as contemplated herein contain, based on their weight, from about 0.01 to about 15 wt. %, preferably from about 0.05 to about 10 wt %, more preferably from about 0.1 to about 7.5 wt. %, and in particular from about 0.15 to about 5 wt. % of at least one proteolipid of formula (II), in which R″ represents keratin or a keratin hydrolyzate.

Further preferred hair treatment agents as contemplated herein contain, based on their weight, from about 0.01 to about 15 wt. %, preferably from about 0.05 to about 10 wt. %, more preferably from about 0.1 to about 7.5 wt. %, and in particular from about 0.15 to about 5 wt. % of at least one proteolipid of formula (II), in which X represents —C(O)—O— or —N⁺(CH₃)₂—CH₂—CH(OH)—CH₂— and R′ represents —(CH₂)₁₁₋₂₃—CH₃, in particular —(CH₂)₁₇—CH₃.

The agents as contemplated herein may contain amino-functional silicones to further enhance the conditioning effects. Preferred agents as contemplated herein contain from about 0.001 to about 50 wt. % of at least one amino-functional silicone.

Particularly preferred agents as contemplated herein contain at least one amino-functional silicone of formula (Si-IIa)

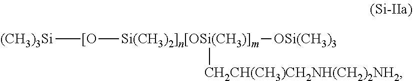

in which m and n are numbers of which the sum (m+n) is between about 1 and about 2000, preferably between about 50 and about 150, n preferably assuming values of from 0 to about 1999 and in particular from about 49 to about 149 and m preferably assuming values of from about 1 to about 2000, in particular from about 1 to about 10.

These silicones are referred to as trimethylsilylamodimethicone according to the INCI declaration.

Also particularly preferred are agents as contemplated herein that contain an amino-functional silicone of formula (Si-IIb)

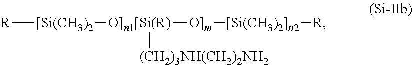

in which R represents —OH, —O—CH₃, or a —CH₃ group and m, n1 and n2 are numbers of which the sum (m+n+n2) is between about 1 and about 2000, preferably between about 50 and about 150, the sum (n1+n2) preferably assuming values of from 0 to about 1999 and in particular from about 49 to about 149 and m preferably assuming values of from about 1 to about 2000, in particular from about 1 to about 10.

These silicones are referred to as amodimethicone according to the INCI declaration.

Irrespective of which amino-functional silicones are used, agents as contemplated herein are preferred that contain an amino-functional silicone of which the amine value is above about 0.25 meq/g, preferably above about 0.3 meq/g, and in particular above about 0.4 meq/g. The amine value here represents the milliequivalents of amine per gram of the amino-functional silicone. Said value can be determined by titration and may also be given in the unit mg KOH/g.

Hair treatment agents which are preferred as contemplated herein contain, based on their weight, from about 0.01 to about 20 wt. %, preferably from about 0.05 to about 10 wt. %, more preferably from about 0.1 to about 7.5 wt. %, and in particular from about 0.15 to about 5 wt. % of amino-functional silicone(s).

The agents as contemplated herein particularly preferably contain amino-functional silicone(s) having terminal hydroxy group(s). Some special amino-functional silicone(s) having terminal hydroxy group(s) have been found to be particularly suitable in the agents as contemplated herein. These are described in the following.

Pretreatment agents that contain at least one silicone of formula (Si-V) have been found to be particularly effective in the method as contemplated herein with regard to the desired effects:

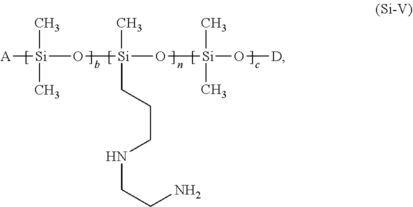

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and about 1000,
   with the proviso that
      n>0 and b+c>0
at least one of the conditions A=—OH or D=—H is met.

Hair treatment agents as contemplated herein that contain, based on their weight, from about 0.01 to about 20 wt. %, preferably from about 0.01 to about 20 wt. %, more preferably from about 0.1 to about 10 wt. %, even more preferably from about 0.5 to about 7.5 wt. %, and in particular from about 0.1 to about 5 wt. % of at least one silicone of formula (Si-V):

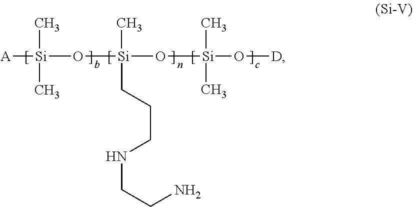

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group;
b, n and c represent integers between 0 and about 1000,
   with the proviso that
      n>0 and b+c>0
      at least one of the conditions A=—OH or D=—H is met,
         are therefore preferred as contemplated herein.

In the above formula (Si-V), the individual siloxane units having the indices b, c and n are randomly distributed, i.e. they are not necessarily block copolymers.

Further particularly suitable silicones are 4-morpholinomethyl-substituted. Hair treatment agents as contemplated herein that contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. % of at least one 4-morpholinomethyl-substituted silicone which comprises structural units of formulas (Si-VIa), (Si-VIb) and (Si-VIc)

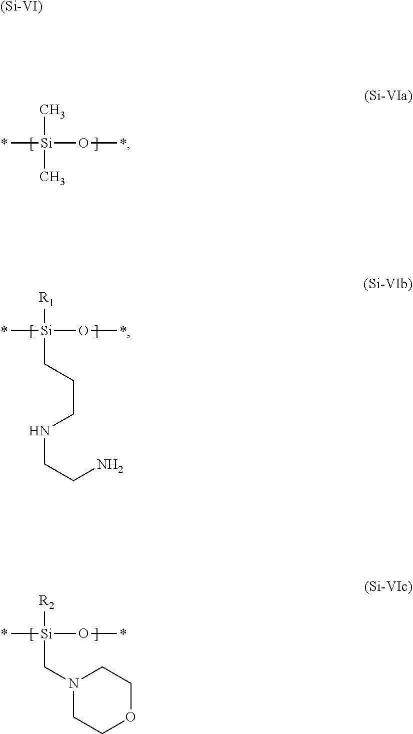

in which

R1 represents —CH₃, —OH, —OCH₃, —O—CH₂CH₃, —O—CH₂CH₂CH₃, or —O—CH(CH₃)₂;
R2 represents —CH₃, —OH, or —OCH₃,
   are particularly preferred.

Particularly preferred hair treatment agents as contemplated herein contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. % of at least one 4-morpholinomethyl-substituted silicone of formula (Si-VI)

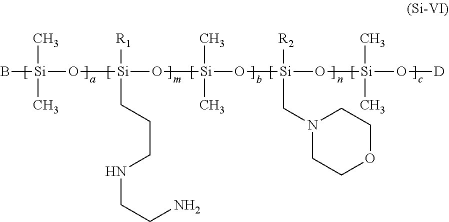

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b and c represent, independently of one another, integers between 0 and about 1000, with the
proviso that a+b+c>0
m and n represent, independently of one another, integers between about 1 and about 1000
with the proviso that
   at least one of the conditions B=—OH or D=—H is met,
   the units a, b, c, m and n are distributed randomly or in blocks in the molecule.

Structural formula (Si-VI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e. the terminal group B or D is preferably bonded to a dimethylsiloxy group. In formula (Si-VI) as well, the siloxane units a, b, c, m and n are preferably distributed randomly.

The silicones represented by formula (Si-VI) and used as contemplated herein can be trimethylsilyl-terminated (D or B=—Si(CH₃)₃), but they may also be dimethylsilylhydroxy-terminated at both ends or dimethylsilylhydroxy-terminated at one end and dimethylsilylmethoxy-terminated at the other end. Within the context of the present disclosure, silicones which are particularly preferably used are selected from silicones in which

B=—O—Si(CH₃)₂OH and D=—Si(CH₃)₃

B=—O—Si(CH₃)₂OH and D=—Si(CH₃)₂OH

B=—O—Si(CH₃)₂OH and D=—Si(CH₃)₂CH₃

B=—O—Si(CH₃)₃ and D=—Si(CH₃)₂OH

B=—O—Si(CH₃)₂OCH₃ and D=—Si(CH₃)₂OH.

These silicones lead to enormous improvements in the hair properties of hair treated with the agents as contemplated herein, and to greatly improved protection in the case of oxidative treatment.

Irrespective of the type of amino-functional silicone(s) having terminal hydroxy group(s) used, the agents as contemplated herein contain the silicone(s) preferably in the form of an emulsion, particularly preferably in the form of a microemulsion.

It has been found that the effect of the silicones used in the agents as contemplated herein can be enhanced further still if certain non-ionic components are also used in the agents. In addition, these non-ionic components have positive effects on the storage stability of the agents. Non-ionic components that are particularly suitable here are ethoxylates of decanol, undecanol, dodecanol, tridecanol, etc. Ethoxylated tridecanols which are particularly preferably incorporated in the agents as contemplated herein have been found to be particularly suitable. Agents which are particularly preferred as contemplated herein contain, based on their weight, from about 0.00001 to about 5 wt. %, preferably from about 0.0001 to about 3.5 wt. %, particularly preferably from about 0.001 to about 2 wt. %, more preferably from about 0.01 to about 1 wt. %, and in particular from about 0.1 to about 0.5 wt. % branched ethoxylated tridecanol (INCI name: trideceth-5) or α-iso-tridecyl-o-hydroxy polyglycol ether (INCI name: trideceth-10) or mixtures thereof.

In addition to or instead of the amino-functional silicone(s), the agents as contemplated herein may contain further silicone(s) which is/are not amino-functional. Hair treatment agents which are preferred as contemplated herein contain, based on their weight, from about 0.01 to about 20 wt. %, preferably from about 0.1 to about 10 wt. %, more preferably from about 0.5 to about 7.5 wt. %, and in particular from about 1 to about 5 wt. % of non-amino-functional silicone(s). Preferred silicones will be described in the following.

Particularly preferred agents as contemplated herein contain at least one silicone of formula Si-I

(CH₃)₃Si—[O—Si(CH₃)₂]ₓ—O—Si(CH₃)₃ (Si-I),

in which x represents a number from 0 to about 100, preferably from 0 to about 50, more preferably from 0 to about 20, and in particular 0 to about 10.

These silicones are referred to as DIMETHICONE, in accordance with INCI nomenclature. The following compounds are preferably used within the context of the present disclosure as a silicone of formula Si-I:

(CH₃)₃Si—O—Si(CH₃)₃, (CH₃)₃Si—O—(CH₃)₂Si—O—Si(CH₃)₃ (CH₃)₃Si—[O—(CH₃)₂Si]₂—O—Si(CH₃)₃

(CH₃)₃Si—[O—(CH₃)₂Si]₃—O—Si(CH₃)₃ (CH₃)₃Si—[O—(CH₃)₂Si]₄—O—Si(CH₃)₃

(CH₃)₃Si—[O—(CH₃)₂Si]₅—O—Si(CH₃)₃ (CH₃)₃Si—[O—(CH₃)₂Si]₆—O—Si(CH₃)₃

(CH₃)₃Si—[O—(CH₃)₂Si]₇—O—Si(CH₃)₃ (CH₃)₃Si—[O—(CH₃)₂Si]₈—O—Si(CH₃)₃

(CH₃)₃Si—[O—(CH₃)₂Si]₉—O—Si(CH₃)₃ (CH₃)₃Si—[O—(CH₃)₂Si]₁₀—O—Si(CH₃)₃

(CH₃)₃Si—[O—(CH₃)₂Si]₁₁—O—Si(CH₃)₃ (CH₃)₃Si—[O—(CH₃)₂Si]₁₂—O—Si(CH₃)₃

(CH₃)₃Si—[O—(CH₃)₂Si]₁₃—O—Si(CH₃)₃ (CH₃)₃Si—[O—(CH₃)₂Si]₁₄—O—Si(CH₃)₃

(CH₃)₃Si—[O—(CH₃)₂Si]₁₅—O—Si(CH₃)₃ (CH₃)₃Si—[O—(CH₃)₂Si]₁₆—O—Si(CH₃)₃

(CH₃)₃Si—[O—(CH₃)₂Si]₁₇—O—Si(CH₃)₃ (CH₃)₃Si—[O—(CH₃)₂Si]₁₈—O—Si(CH₃)₃

(CH₃)₃Si—[O—(CH₃)₂Si]₁₉—O—Si(CH₃)₃ (CH₃)₃Si—[O—(CH₃)₂Si]₂₀—O—Si(CH₃)₃

(CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ being particularly preferred.

Of course, mixtures of the above-mentioned silicones can also be contained in the agents as contemplated herein. Preferred silicones that can be used as contemplated herein have viscosities at about 20° C. of from about 0.2 to about 2 mm²s⁻¹, silicones having viscosities of from about 0.5 to about 1 mm²s⁻¹ being particularly preferred.

The hair treatment agents as contemplated herein may contain surfactant(s). In cleaning compositions (shampoos), in particular anionic surfactants have been found to be suitable and, in conditioning compositions, cationic surfactants are ingredients that are often used; owing to their advantageous properties, amphoteric surfactants are particularly preferably used in both shampoos and conditioners.

The hair treatment agents as contemplated herein may contain at least one anionic surfactant.

Suitable anionic surfactants and emulsifiers for the compositions as contemplated herein include all anionic surface-active substances suitable for use on the human body. These are exemplified by a water-solubilizing, anionic group such as a carboxylate, sulfate, sulfonate or phosphate group and a lipophilic alkyl group having approximately 8 to 30 C atoms. In addition, glycol or polyglycol ether groups, ester, ether and amide groups, and hydroxyl groups, may additionally be contained in the molecule.

Preferred hair treatment agents contain, based on their weight, from about 0.5 to about 20 wt. %, preferably from about 0.75 to about 15 wt. %, more preferably from about 1 to about 12 wt. %, and in particular from about 2 to about 10 wt. % of anionic surfactant(s).

Particularly preferred hair treatment agents as contemplated herein contain, based on their weight, from about 0.5 to about 20 wt. %, preferably from about 0.75 to about 15 wt. %, more preferably from about 1 to about 12 wt. %, and in particular from about 2 to about 10 wt. %, alkyl (ether) sulfates of general formula R—(OCH₂—CH₂)ₙ—OSO₃X, in which R is a straight-chain or branched, saturated or unsaturated alkyl group having 8 to 24 C atoms, n is the number 0 or from about 1 to about 12, and X is an alkali, alkaline-earth, ammonium or alkanolamine ion.

The hair treatment agents as contemplated herein may contain at least one amphoteric surfactant and/or at least one non-ionic surfactant.

Particularly preferred hair treatment agents as contemplated herein contain, based on their weight, from about 0.3 to about 20 wt. %, preferably from about 0.5 to about 8 wt. %, more preferably from about 0.75 to about 6 wt. %, and in particular from about 1 to about 5 wt. % amphoteric surfactant(s).

Surfactants that have both a negatively-charged functional group and a positively-charged functional group are referred to as amphoteric surfactants or zwitterionic surfactants.

Particularly preferred hair treatment agents contain, as amphoteric surfactants, betaines of formula (Bet-I)

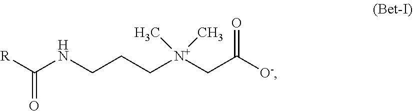

in which R represents a straight-chain or branched, saturated or mono- or polyunsaturated alkyl or alkenyl functional group having 8 to 24 carbon atoms.

In accordance with INCI nomenclature, these surfactants are referred to as amidopropyl betaines, the representatives derived from coconut fatty acids being preferred and referred to as cocamidopropyl betaines. It is particularly preferable as contemplated herein to use surfactants of formula (Bet-I) that are a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—(O)—NH—(CH₂)₃N⁺(CH₃)₂CH₂COO⁻

Particularly preferably, surfactants of formula (Bet-I) are used within narrower amount ranges. In this case, hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. % of surfactant(s) of formula (Bet-I) are preferred.

In addition to or instead of the ampho-surfactant(s) of formula (Bet-I), the hair treatment agents as contemplated herein may particularly preferably contain, as amphoteric surfactants, betaines of formula (Bet-II)

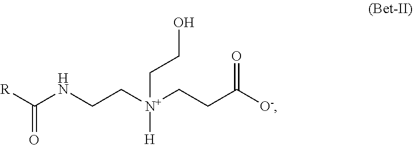

in which R represents a straight-chain or branched, saturated or mono- or polyunsaturated alkyl or alkenyl functional group having 8 to 24 carbon atoms.

In accordance with INCI nomenclature, these surfactants are referred to as amphoacetates, the representatives derived from coconut fatty acids being preferred and referred to as cocoamphoacetates.

For production reasons, surfactants of this type always also contain betaines of formula (Bet-IIa)

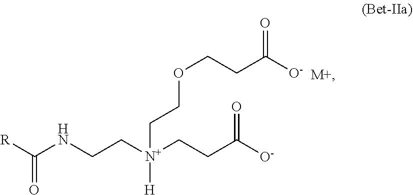

in which R represents a straight-chain or branched, saturated or mono- or polyunsaturated alkyl or alkenyl functional group having 8 to 24 carbon atoms, and M represents a cation.

In accordance with INCI nomenclature, these surfactants are referred to as amphodiacetates, the representatives derived from coconut fatty acids being preferred and referred to as cocoamphodiacetates.

It is particularly preferable as contemplated herein to use surfactants of formula (Bet-II) that are a mixture of the following representatives:

H₃C—(CH₂)₇—C(O)—NH—(CH₂)₂NH⁺(CH₂CH₂OH)CH₂CH₂COO⁻

H₃C—(CH₂)₉—C(O)—NH—(CH₂)₂NH⁺(CH₂CH₂OH)CH₂CH₂COO⁻

H₃C—(CH₂)₁₁—C(O)—NH—(CH₂)₂NH⁺(CH₂CH₂OH)CH₂CH₂COO⁻

H₃C—(CH₂)₁₃—C(O)—NH—(CH₂)₂NH⁺(CH₂CH₂OH)CH₂CH₂COO⁻

H₃C—(CH₂)₁₅—C(O)—NH—(CH₂)₂NH⁺(CH₂CH₂OH)CH₂CH₂COO⁻

H₃C—(CH₂)₇—CH═CH—(CH₂)₇—C(O)—NH—(CH₂)₂NH⁺(CH₂CH₂OH)CH₂CH₂COO⁻

Particularly preferably, surfactants of formula (Bet-II) are used within narrower amount ranges. In this case, hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. % surfactant(s) of formula (Bet-II) are preferred.

In summary, cosmetic agents as contemplated herein are preferred in which the functional group R in formulas (Bet-I) and (Bet-II) is selected from H₃C—(CH₂)₇—H₃C—(CH₂)₉—H₃C—(CH₂)₁₁—H₃C—(CH₂)₁₃—H₃C—(CH₂)₁₅—H₃C—(CH₂)₇—CH═CH—(CH₂)₇— or mixtures thereof.

The hair treatment agents may contain non-ionic surfactant(s).

Hair treatment agents which are preferred as contemplated herein contain, based on their weight, from about 0.3 to about 10 wt. %, preferably from about 0.5 to about 8 wt. %, more preferably from about 0.75 to about 6 wt. %, and in particular from about 1 to about 5 wt. % non-ionic surfactant(s).

The hair treatment agents may contain cationic surfactant(s). As contemplated herein, it is possible to use cationic surfactants of the following types: quaternary ammonium compounds, esterquats, and amidoamines. Preferred quaternary ammonium compounds are ammonium halides, in particular chlorides and bromides, such as alkyl trimethyl ammonium chlorides, dialkyl dimethyl ammonium chlorides, and trialkyl methyl ammonium chlorides. The long alkyl chains of these surfactants preferably have 10 to 18 carbon atoms, such as in cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride. Further preferred cationic surfactants are the imidazolium compounds known by the INCI names quaternium-27 and quaternium-83.

Particularly preferred hair treatment agents as contemplated herein contain, as the cationic care substance, based on their weight, from about 0.05 to about 7.5 wt. %, preferably from about 0.1 to about 5 wt. %, particularly preferably from about 0.2 to about 3.5 wt. %, and in particular from about 0.25 to about 2.5 wt. % cationic surfactant(s) from the group of quaternary ammonium compounds and/or esterquats and/or amidoamines.

Particularly preferred hair treatment agents as contemplated herein contain, based on the weight of the agent, from about 0.05 to about 20 wt. %, preferably from about 0.1 to about 10 wt. %, more preferably from about 0.25 to about 8 wt. %, and in particular from about 0.5 to about 7 wt. % cationic surfactant(s), preferably from about 0.05 to about 20 wt. %, more preferably from about 0.1 to about 10 wt. %, even more preferably from about 0.25 to about 8 wt. %, and in particular from about 0.5 to about 7 wt. % behenyl trimethyl ammonium chloride.

The agents as contemplated herein may contain at least one cationic polymer.

Cationic polymers enhance the nourishing performance of the hair treatment agents as contemplated herein (in particular the effectiveness of the agents as contemplated herein with respect to hair breakage).

Irrespective of which cationic polymer(s) is/are used, preferred hair treatment agents contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. % cationic polymer(s).

It is especially preferable as contemplated herein to use cationic polysaccharide polymers as the cationic polymers.

Cationic polysaccharide polymers enhance the nourishing performance of the hair treatment agents as contemplated herein (in particular the effectiveness of the agents as contemplated herein with respect to hair breakage). Suitable cationic polysaccharide polymers may be selected from cationic cellulose compounds and/or from cationic guar derivatives.

Particularly preferred hair treatment agents as contemplated herein contain, as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. % of at least one polymer from the group of cationic cellulose polymers and/or cationic guar derivatives.

Cationic cellulose compounds within the meaning of the present disclosure are those that carry more than one permanent cationic charge in at least one side chain. Cellulose is constructed from beta-1,4-glycosidically linked D-glucopyranose units and forms unbranched, water-insoluble chains. A “side chain” of cellulose is defined as chemical substituents which bind to the cellulose backbone and are bit part of the native cellulose, since they have been introduced subsequently, for example by chemical synthesis.

Quaternized cellulose polymers originating from hydroxy(C₂-C₄)alkyl celluloses, particularly preferably from hydroxyethyl celluloses, are preferred.

Polymers of this kind are known to a person skilled in the art and are commercially available from various companies. The cationic cellulose derivatives known by the INCI names polyquarternium-4, polyquarternium-10, polyquaternium-24, polyquarternium-67, and/or polyquaternium-72 are particularly preferred. Polyquaternium-10, polyquarternium-24, and/or polyquarternium-67 are very particularly preferred, with polyquarternium-10 being particularly preferred.

Preferred hair treatment agents as contemplated herein contain, as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. % of at least one polymer from the group of polyquarternium-4, polyquarternium-10, polyquarternium-24, polyquarternium-67, and/or polyquarternium-72.

Particularly preferred hair treatment agents as contemplated herein contain, as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. % polyquarternium-10.

Suitable cationic guar derivatives within the meaning of the present disclosure are cationic hydroxyalkyl guar derivatives, preferably cationic hydroxyethyl trimethyl ammonium guar and/or cationic hydroxypropyl trimethyl ammonium guar having average molecular weights between about 100,000 and about 2,000,000 daltons. The cationic guar polymers known by the INCI name guar hydroxypropyltrimonium chloride having a molecular weight (weight average) between about 200,000 and about 1,600,000 daltons are particularly preferred. The cationic charge density of these guar polymers is preferably at least about 0.4 meq/g, more preferably at least about 0.5 meq/g, and in particular at least about 0.6 meq/g. The nitrogen content thereof is preferably in the range of from about 1.1 to about 1.8 wt. % (based on the total weight thereof).

Cationic guar derivatives known by the INCI name guar hydroxypropyltrimonium chloride are known to a person skilled in the art and are obtainable from various providers under the trade names Cosmedia® Guar, N-Hance® and/or Jaguar®, for example.

Particularly preferred hair treatment agents as contemplated herein contain, as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, guar hydroxypropyltrimonium chloride.

In summary, hair treatment agents which are preferred as contemplated herein contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. % cationic polymer(s), and preferably from about 0.01 wt. % to about 3 wt. %, more preferably from about 0.05 to about 2 wt. %, even more preferably from about 0.1 to about 1.5 wt. %, and in particular from about 0.15 to about 0.8 wt. % of at least one polymer from the group of cationic cellulose polymers and/or cationic guar derivatives.

It has been found that alpha-substituted aldehydes can further enhance the effect of the agents as contemplated herein. Significant increases in performance are observed, in particular with regard to the inner structural strengthening of keratin fibers and the prevention of washout.

Hair treatment agents which are preferred as contemplated herein additionally contain, based on the weight of the agent, from about 0.001 to about 20 wt. % of at least one alpha-substituted aldehyde.

Preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. % alpha-substituted aldehyde(s) of formula (I)

Y—CH(X)—CHO (I),

in which
X represents —OH, —Cl, —Br, —I, —O—(CH₂)ₙ—CH₃, where n=1, 2, 3 or 4, or —O—(CH₂)ₘ—OH, where m=1, 2 or 3,
Y represents —H, or —CH₃ or H₃C—(CH₂)ₖ—, where k=1, 2, 3, 4, 5, 6, 7, 8 or —OH or —(CH₂)ₚ—OH, where p=1, 2, 3, 4 or —CHO.

Aldehydes that are preferably to be used as contemplated herein are described on pages 39 to 42 of the priority document as numbers 1 to 176.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. % of at least one alpha-substituted aldehyde from the group
2-hydroxypropanal (X=—OH, Y=—CH₃):
2-hydroxyhexanal (X=—OH, Y=H₃C—(CH₂)ₖ—, where k=3):
2-hydroxyoctanal (X=—OH, Y=H₃C—(CH₂)ₖ—, where k=5):
bromomalonaldehyde (X=Br, Y=—CH═):
2-(2-hydroxyethoxy)acetaldehyde (X=—O—(CH₂)ₚ—OH, where p=2, Y=—H)
glyceraldehyde (X=—OH, Y=—(CH₂)ₚ—OH, where p=1).

Complexing agents can further enhance the effect of the agents as contemplated herein, complexing agents which are derived from polycarboxylic acids having been found to be particularly suitable.

Hair treatment agents which are preferred as contemplated herein contain, based on their weight, from about 0.001 to about 20 wt. % complexing agents from the group tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriacetic acid (HEDTA).

The use of the three complexing agents mentioned first is very particularly preferred. Extremely preferred hair treatment agents as contemplated herein contain, based on their weight, from about 0.001 to about 20 wt. %, preferably from about 0.005 to about 15 wt. %, more preferably from about 0.01 to about 10 wt. %, even more preferably from about 0.05 to about 7.5 wt. %, and in particular from about 0.1 to about 5 wt. % complexing agents from the group
a. tetrasodium-N,N-bis(carboxylatomethyl)-L-glutamate (tetrasodium glutamate diacetate, GLDA),

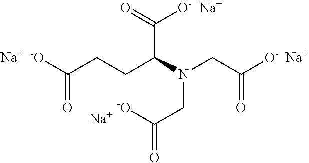

b. pentasodium diethylenetriaminepentaacetate (DTPA)

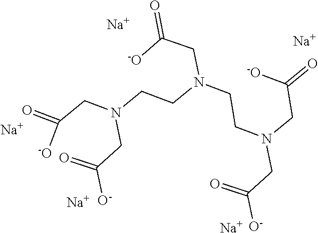

c. tetrasodium iminodisuccinate (IDS).

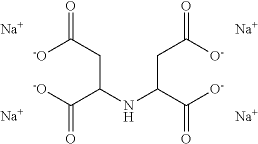

The agents as contemplated herein may contain only one of the three complexing agents mentioned. However, it is also possible for the agents as contemplated herein to contain two or all three of the above-mentioned complexing agents, the amount of all the complexing agents from the above-mentioned group contained in the agents being within an amount range of from about 0.01 to about 20 wt. %.

Hair treatment agents which are preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. % tetrasodium-N,N-bis(carboxylatomethyl)-L-glutamate (tetrasodium glutamate diacetate, GLDA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. % of pentasodium diethylenetriaminepentaacetate (DTPA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. % of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. % of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. % of pentasodium diethylenetriaminepentaacetate (DTPA).

Hair treatment agents which are also preferred as contemplated herein contain, based on their weight, from about 0.01 to 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. % of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. % of pentasodium diethylenetriaminepentaacetate (DTPA), and from about 0.01 to about 10 wt. %, more preferably from about 0.05 to about 7.5 wt. %, and in particular from about 0.1 to about 5 wt. % of tetrasodium iminodisuccinate (IDS).

Hair treatment agents which are also preferred as contemplated herein contain, based on their weight, from about 0.1 to about 5 wt. % tetrasodium-N,N-bis(carboxylatomethyl)-L-glutamate (tetrasodium glutamate diacetate, GLDA), from about 0.1 to about 5 wt. % pentasodium diethylenetriaminepentaacetate (DTPA), and from about 0.1 to about 5 wt. % tetrasodium iminodisuccinate (IDS).

The hair treatment agents may contain at least one bivalent or trivalent metal salt. These lead to improved washout prevention. To achieve an optimum effect, it is advantageous for the metal salts to be present in dissolved form in the agents as contemplated herein.

In a preferred embodiment, the hair cleaning and care agents as contemplated herein therefore contain water-soluble bivalent or trivalent metal salts. “Water-soluble” is understood here to mean that, at about 20° C., at least about 1 g of the salt in question can dissolve fully in about 1 L of water.

Suitable bivalent or trivalent metal salts may be selected from bivalent or trivalent organic and/or inorganic salts.

Particularly suitable cations within these salts may preferably be selected from alkaline-earth metal cations and from copper, zinc, iron(II), iron(III), and/or aluminum cations. Alkaline-earth metal cations, and particularly preferably calcium and magnesium cations, are very particularly preferred. Particularly suitable organic anions within these salts may preferably be selected from formate, acetate, lactate, succinate, citrate, tartrate, malate, maleate, oxalate, and/or glycolate ions. Acetate, lactate, and/or citrate salts having the aforementioned cations are very particularly preferred.

Particularly preferred organic salts are calcium lactate, calcium citrate, calcium acetate, magnesium lactate, magnesium citrate, and/or magnesium acetate.

Particularly suitable inorganic anions within these salts may be selected from halide, sulfate, phosphate, and/or carbonate ions. Sulfate and/or halide ions such as chloride and bromide ions are very particularly preferred.

Particularly preferred inorganic salts are calcium chloride, calcium sulfate, magnesium chloride, and/or magnesium sulfate.

The weight proportion of the at least one bivalent or trivalent metal salt with respect to the total weight of the hair treatment agents as contemplated herein is preferably from about 0.01 to about 10 wt. %, more preferably from about 0.1 to about 7.5 wt. %, even more preferably from about 0.2 to about 5 wt. %, and in particular from about 0.3 to about 3 wt. %.

Hair treatment agents which are preferred as contemplated herein contain, based on their weight, from about 0.01 to about 10 wt. %, preferably from about 0.1 to about 7.5 wt. %, more preferably from about 0.2 to about 5 wt. %, and in particular from about 0.3 to about 3 wt. % of at least one bivalent or trivalent metal salt from the group of organic or inorganic copper, zinc, iron(II), calcium, magnesium, iron(III), and/or aluminum salts.

Water-soluble salts are particularly preferred in this embodiment. Calcium lactate, calcium citrate, calcium acetate, magnesium lactate, magnesium citrate, magnesium acetate, calcium halides, calcium hydroxide, magnesium halides, and/or magnesium hydroxide are very particularly preferred in this embodiment.

The hair treatment agents contain the above-described active ingredients preferably in a cosmetically acceptable carrier. Within the context of the present disclosure, this is understood to preferably mean an aqueous or aqueous-alcoholic carrier.

The cosmetic carrier preferably contains at least about 50 wt. %, more preferably at least about 60 wt. %, particularly preferably at least about 70 wt. %, and more particularly preferably at least about 75 wt. % water.

Furthermore, the cosmetic carrier may contain from about 0.01 to about 40 wt. %, preferably from about 0.05 to about 30 wt. %, and in particular from about 0.1 to about 20 wt. % of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of said alcohols.

Water-soluble alcohols are particularly preferred. Ethanol, 1,2-propylene glycol, glycerol, benzyl alcohol, and mixtures of said alcohols are particularly preferred.

It is advantageous for very good (scalp) skin compatibility of the hair treatment agents as contemplated herein if said agents have a slightly acidic pH.

It has been found that the agents as contemplated herein have particularly good skin compatibility and mildness in a pH range of from about 4.2 to about 5.8.

In a first preferred embodiment, the hair treatment agents as contemplated herein therefore preferably have a pH in the range of from about 4.2 to about 5.8, more preferably from about 4.3 to about 5.6, particularly preferably from about 4.4 to about 5.5, extremely preferably from about 4.5 to about 5.4, and particularly preferably from about 4.7 to about 5.3.

The hair treatment agents as contemplated herein may contain vegetable oils, vegetable butters, and/or vegetable waxes. These vegetable oil components give the hair improved combability and stylability and increase hair shine.

Suitable vegetable oil components include natural (vegetable) oils and/or butters which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Carnauba wax, beeswax, and/or candelilla wax can preferably be used as suitable natural or vegetable waxes.

The weight proportion of the at least one vegetable oil, vegetable butter, and/or vegetable wax with respect to the total weight of the hair treatment agents as contemplated herein is preferably from about 0.02 to about 2.50 wt. %, more preferably from about 0.03 to about 2.00 wt. %, particularly preferably from about 0.04 to about 1.50 wt. %, and in particular from about 0.05 to about 1.00 wt. %.

In addition to the aforementioned essential and optional components, the hair treatment agents as contemplated herein may contain, in a further preferred embodiment for further enhancing the nourishing properties of the agents, at least one further hair-conditioning active ingredient, which can be selected from the group of
protein hydrolyzates,
vitamins,
plant extracts, and/or
glycerol.

Suitable protein hydrolyzates are understood to mean product mixtures that can be obtained by acid, base, or enzyme-catalyzed degradation of proteins.

Protein hydrolyzates of plant, animal, and/or marine origin can be used.

Animal protein hydrolyzates are, for example, elastin, collagen, keratin, silk, and milk protein hydrolyzates, which can also be present in the form of salts. Such products are marketed, for example, under the trademarks Dehylan® (Cognis), Promois® (Interorgana), Collapuron® (Cognis), Nutrilan® (Cognis), Gelita-Sol* (Deutsche Gelatine Fabriken Stoess & Co), Lexein® (Inolex), and Kerasol® (Croda).

Protein hydrolyzates of plant origin are preferred, for example soybean, almond, rice, pea, potato and wheat protein hydrolyzates. Such products are available, for example, under the trademarks Gluadin® (Cognis), DiaMin® (Diamalt), Lexein® (Inolex), and Crotein® (Croda). Cationized protein hydrolyzates can also be used, it being possible for the underlying protein hydrolyzate to originate from animals, for example from collagen, milk or keratin, from plants, for example from wheat, corn, rice, potatoes, soybeans or almonds, from marine life forms, for example from fish collagen or algae, or from biotechnologically obtained protein hydrolyzates. The protein hydrolyzates forming the basis of the cationic derivatives may be obtained from the corresponding proteins by chemical, in particular alkaline or acidic, hydrolysis, by enzymatic hydrolysis, and/or by a combination of both types of hydrolysis. The hydrolysis of proteins usually results in a protein hydrolyzate having a molecular weight distribution of approximately 100 daltons up to several thousand daltons. Cationic protein hydrolyzates of which the underlying protein portion has a molecular weight of from about 100 to about 25,000 daltons, preferably from about 250 to about 5,000 daltons, are preferred. Furthermore, cationic protein hydrolyzates are understood to mean quaternized amino acids and mixtures thereof. Quaternization of the protein hydrolyzates or of the amino acids is often carried out by means of quaternary ammonium salts such as, for example, N,N-dimethyl-N-(n-alkyl)-N-(2-hydroxy-3-chloro-n-propyl)ammonium halides. Furthermore, the cationic protein hydrolyzates can also be derivatized even further.

The weight proportion of the protein hydrolyzate(s) with respect to the total weight of the hair treatment agents is preferably from about 0.01 to about 5 wt. %, more preferably from about 0.025 to about 3 wt. %, and in particular from about 0.05 to about 2 wt. %.

Irrespective of the source (plant, animal, marine, etc.), protein hydrolyzates contain, depending on the degree of hydrolysis, individual amino acids, oligopeptides, and optionally polypeptides.

The hair treatment agents as contemplated herein particularly preferably contain at least one oligopeptide that has at least one amino acid sequence Glu-Glu-Glu, it being possible for the amino group to be present in a free or protonated form and for the carboxy groups to be present in a free or deprotonated form.

Preferred hair treatment agents as contemplated herein contain, based on their weight, from about 0.0001 to about 10 wt. % of at least one oligopeptide that has at least one amino acid sequence Glu-Glu-Glu, it being possible for the amino group to be present in a free or protonated form and for the carboxy groups to be present in a free or deprotonated form.

In this formula, as in all preceding formulas, the bracketed hydrogen atom of the amino group, as well as the bracketed hydroxy group of the acid function, means that the groups in question can be present as such (making it an oligopeptide having the relevant number of amino acids, as shown in formula 3 above), or that the amino acid sequence is present in an oligopeptide which also comprises further amino acids; depending on where the further amino acid(s) is/are bound, the bracketed components of the above-mentioned formula are replaced by the further amino acid functional group(s).

These preferred hair treatment agents as contemplated herein contain, based on their weight, from about 0.0001 to about 10 wt. % of at least one oligopeptide that has at least one amino acid sequence Glu-Glu-Glu, i.e. at least three consecutive glutamic acids.

Within the meaning of the present application, oligopeptides are condensation products of amino acids which are linked by peptide bonds in the manner of an acid amide and which comprise at least about 3 and at most about 25 amino acids.

In hair treatment agents which are preferred as contemplated herein, the oligopeptide comprises from about 5 to about 15 amino acids, preferably from about 6 to about 13 amino acids, particularly preferably from about 7 to about 12 amino acids, and in particular about 8, about 9 or about 10 amino acids.

Depending on whether further amino acids are bound to the sequence Glu-Glu-Glu and depending on the type of these amino acids, the molar mass of the oligopeptide contained in the agents as contemplated herein may vary. Hair treatment agents which are preferably used as contemplated herein are exemplified in that the oligopeptide has a molar mass of from about 650 to about 3,000 daltons, preferably from about 750 to about 2,500 daltons, particularly preferably from about 850 to about 2,000 daltons, and in particular from about 1,000 to about 1,600 daltons.

In summary, preferred hair treatment agents are exemplified in that the oligopeptide comprises from about 5 to about 15 amino acids, preferably from about 6 to about 13 amino acids, particularly preferably from about 7 to about 12 amino acids, and in particular about 8, about 9 or about 10 amino acids, and has a molar mass of from about 650 to about 3,000 daltons, preferably from about 750 to about 2,500 daltons, particularly preferably from about 850 to about 2,000 daltons, and in particular from about 1,000 to about 1,600 daltons.

As can be seen from the preferred number of amino acids in the oligopeptides and the preferred molar mass range, oligopeptides are preferably used which consist not only of the three glutamic acids but also have additional amino acids bound to this sequence. These further amino acids are preferably selected from certain amino acids, whereas certain other representatives are less preferred as contemplated herein.

For instance, it is preferred if the oligopeptides used in the agents as contemplated herein do not contain any methionine.

It is further preferred if the oligopeptides used in the agents as contemplated herein do not contain any cysteine and/or cystine. It is further preferred if the oligopeptides used in the agents as contemplated herein do not contain any aspartic acid and/or asparagine. It is further preferred if the oligopeptides used in the agents as contemplated herein do not contain any serine and/or threonine. Particularly preferred oligopeptides or amino acid sequences contained in the preferred oligopeptides are described below:

A particularly preferred oligopeptide additionally contains tyrosine, which is preferably bound via its acid function to the sequence Glu-Glu-Glu. Hair treatment agents that are preferred as contemplated herein are therefore exemplified in that the oligopeptide contained therein has at least one amino acid sequence Tyr-Glu-Glu-Glu, it being possible for the amino group to be present in a free or protonated form and for the carboxy groups to be present in a free or deprotonated form.

A further particularly preferred oligopeptide additionally contains isoleucine, which is preferably bound via its amino function to the sequence Glu-Glu-Glu. Hair treatment agents which are preferred as contemplated herein are therefore exemplified in that the oligopeptide contained therein has at least one amino acid sequence Glu-Glu-Glu-Ile, it being possible for the amino group to be present in a free or protonated form and for the carboxy groups to be present in a free or deprotonated form.

Oligopeptides that have both of the aforementioned amino acids (tyrosine and isoleucine) are preferred as contemplated herein. In this case, hair treatment agents as contemplated herein are particularly preferred in which the oligopeptide contained in the hair treatment agent has at least one amino acid sequence Tyr-Glu-Glu-Glu-Ile, it being possible for the amino group to be present in a free or protonated form and for the carboxy groups to be present in a free or deprotonated form.

Further preferred oligopeptides additionally contain arginine, which is preferably present bound to isoleucine.

Particularly preferred in this case are hair treatment agents as contemplated herein in which the oligopeptide contained in the hair treatment agent has at least one amino acid sequence Tyr-Glu-Glu-Glu-Ile-Arg, it being possible for the amino groups to be present in a free or protonated form and for the carboxy groups to be present in a free or deprotonated form.

Still further preferred oligopeptides additionally contain valine, which is preferably present bound to the arginine. Hair treatment agents that are further preferred as contemplated herein are therefore exemplified in that the oligopeptide contained in the hair treatment agent has at least one amino acid sequence Tyr-Glu-Glu-Glu-Ile-Arg-Val, it being possible for the amino groups to be present in a free or protonated form and for the carboxy groups to be present in a free or deprotonated form.

Still further preferred oligopeptides additionally contain leucine which is preferably present bound to the valine. Hair treatments agents that are further preferred as contemplated herein are therefore exemplified in that the oligopeptide contained in the hair treatment agent has at least one amino acid sequence Tyr-Glu-Glu-Glu-Ile-Arg-Val-Leu, it being possible for the amino groups to be present in a free or protonated or form and for the carboxy groups to be present in a free or deprotonated form.

Particularly preferred oligopeptides additionally contain leucine which is preferably present bound to the tyrosine. Hair treatment agents that are further preferred as contemplated herein are therefore exemplified in that the oligopeptide contained in the hair treatment agent has at least one amino acid sequence Leu-Tyr-Glu-Glu-Glu-Ile-Arg-Val-Leu, it being possible for the amino groups to be present in a free or protonated form and for the carboxy groups to be present in a free or deprotonated form.

It is very particularly preferred that the highest possible proportion of all the keratin peptides contained in the agent as contemplated herein meets the above-mentioned conditions.

Preferred hair treatment agents as contemplated herein are exemplified in that at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. % of all the keratin peptides contained in the agent have the amino acid sequence Glu-Glu-Glu.

Particularly preferred hair treatment agents as contemplated herein are exemplified in that at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. % of all the keratin peptides contained in the agent have the amino acid sequence Tyr-Glu-Glu-Glu-Ile.

Particularly preferred hair treatment agents as contemplated herein are exemplified in that at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. % of all the keratin peptides contained in the agent have the amino acid sequence Tyr-Glu-Glu-Glu-Ile-Arg-Val-Leu.

The aforementioned conditions relate to the total content of peptides originating from keratin materials in the agents as contemplated herein. In addition to the oligopeptides of keratin origin, further peptides and/or protein hydrolyzates can of course be used, for example from other native sources. For example, the additional use of wheat protein hydrolyzates is preferred.

The weight proportion of the vitamin(s), vitamin derivative(s), and/or the vitamin precursor(s) with respect to the total weight of the hair treatment agents is preferably from about 0.001 to about 2 wt. %, particularly preferably from about 0.005 to about 1 wt. %, and in particular from about 0.01 to about 0.5 wt. %.

The plant extracts can be used in the hair treatment agents as contemplated herein (based on the total weight of the agents) preferably in an amount of from about 0.01 to about 10 wt. %, more preferably from about 0.05 to about 7.5 wt. %, and in particular from about 0.1 to about 5 wt. %.

Glycerol can be added to the hair cleaning and care agents separately in an amount of up to about 10 wt. % (based on the total weight of the agent). However, it may also be a component of the above-mentioned aqueous-alcoholic carrier.

It has been found that the hair treatment agents as contemplated herein are also suitable for use as an anti-dandruff preparation.

The total weight of anti-dandruff agents with respect to the total weight of the hair treatment agents can preferably be from about 0.01 to about 10 wt. %, more preferably from about 0.025 to about 7.5 wt. %, particularly preferably from about 0.05 to about 5 wt. %, and in particular from about 0.075 to about 3 wt. %.

Suitable anti-dandruff active ingredients can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione and piroctone olamine are preferred.

The agents as contemplated herein can be formulated as what are referred to as rinse-off products, i.e. are rinsed out of the hair again after a specific contact time. This contact time is preferably less than an hour, i.e. the user preferably does not leave the products in the hair until the next time the hair is washed.

The present disclosure therefore further relates to a method for treating hair in which an agent as contemplated herein is applied to dry or wet hair, is left there for a period of from about 10 to about 300 seconds, and is subsequently rinsed out.

The agents as contemplated herein can also be formulated as what are referred to as leave-on products, i.e. are not rinsed out of the hair, but are instead left there until the next time the hair is washed.

The present disclosure therefore further relates to a method for treating hair in which an agent as contemplated herein is applied to dry or wet hair and is left there until the next time the hair is washed.

The agents as contemplated herein lead to significantly increased strengthening of the internal and external hair structure. The present disclosure therefore further relates to the use of agents as contemplated herein for strengthening the hair structure, in particular the internal hair structure.

Within the meaning of the present disclosure, “structure strengthening” is understood to mean a reduction in the damage to keratin fibers caused by a wide range of influences. Restoring the natural strength, for example, plays a significant role in this.

Restructured fibers are distinguished, for example, by improved shine, by an improved feel, and by easier combability. In addition, they have optimized strength and elasticity. Successful structure strengthening or restructuring can be verified physically as an increase in the melting point in comparison with damaged fibers.

The agents as contemplated herein also lead to considerably increased stability of artificial colors against the washout of color. Chemically colored hair can thus be washed much more frequently using the agents as contemplated herein, without this leading to undesired bleeding or fading of the color.

The present disclosure therefore further relates to the use of agents as contemplated herein for reducing the washout of color from chemically colored hair.

The statements made concerning the agents as contemplated herein apply, mutatis mutandis, to particularly preferred embodiments of the methods as contemplated herein and of the uses as contemplated herein.

### Examples

All values in wt. %.
Succinimidyl esters used:

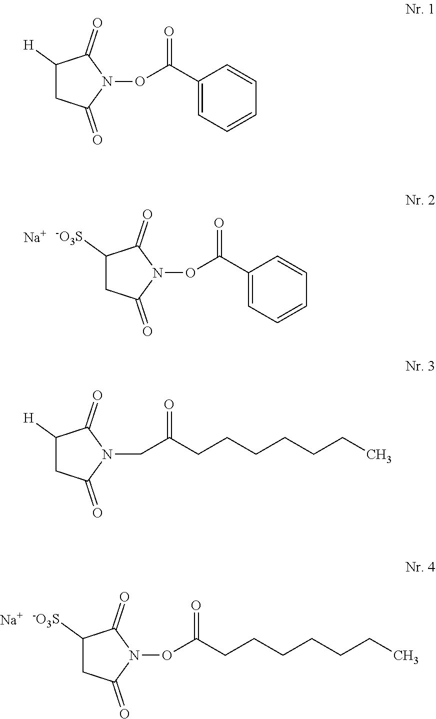

Hair shampoo:

| | 1 | 2 | 3 | 4 | 5 | 6 |
|---|---|---|---|---|---|---|
| Sodium laureth sulfate | 10 | 10 | 10 | 10 | 10 | 10 |
| Cocamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| Succinimidyl ester no. 1 | 0.1 | — | — | — | 0.1 | — |
| Succinimidyl ester no. 2 | | 0.15 | | | | |
| Succinimidyl ester no. 3 | — | — | 0.1 | — | — | 0.1 |
| Succinimidyl ester no. 4 | — | — | — | 0.15 | — | — |
| Glyceraldehyde | — | — | — | — | 0.1 | — |
| Bromomalonaldehyde | — | — | — | — | — | 0.2 |
| Panthenol | 0.5 | 0.5 | 0.5 | 0.5 | 0.5 | 0.5 |
| 2-hydroxyoctanal | 1.0 | 1.0 | 1.0 | 1.0 | 1.0 | 1.0 |
| Cocodimonium | 1.0 | 2.0 | 3.5 | 1.0 | 0.8 | 1.0 |
| hydroxypropyl | | | | | | |
| hydrolyzed keratin | | | | | | |
| Laureth-2 | 1.2 | 1.2 | 1.2 | 1.2 | 1.2 | 1.2 |
| Sodium chloride | 1.3 | 1.0 | 1.0 | 1.2 | 1.1 | 1.3 |
| Water, preservative, | up to | up to | up to | up to | up to | up to |
| perfume oils | 100 | 100 | 100 | 100 | 100 | 100 |

Hair conditioner:

| | 1 | 2 | 3 | 4 | 5 | 6 |
|---|---|---|---|---|---|---|
| Quatemium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar | 0.2 | 0.2 | 0.2 | 0.2 | 0.2 | 0.2 |
| hydroxypropyltrimonium | | | | | | |
| chloride | | | | | | |
| Polyquatemium-37 | 0.4 | 0.4 | 0.4 | 0.4 | 0.4 | 0.4 |
| Steardimonium hydroxypropyl | 0.1 | 0.1 | 0.1 | 0.1 | 0.1 | 0.1 |
| hydrolyzed keratin | | | | | | |
| Shea butter | 1.5 | 1.5 | 1.5 | 1.5 | 1.5 | 1.5 |
| Behenoyl PG-trimonium | 1.5 | 1.5 | 1.5 | 1.5 | 1.5 | 1.5 |
| chloride | | | | | | |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Lactic acid | 0.1 | 0.1 | 0.1 | 0.1 | 0.1 | 0.1 |
| Succinimidyl ester no. 1 | 0.1 | — | — | — | — | — |
| Succinimidyl ester no. 2 | — | 0.15 | — | — | 0.1 | — |
| Succinimidyl ester no. 3 | — | — | 0.1 | — | — | — |
| Succinimidyl ester no. 4 | — | — | — | 0.15 | — | 0.1 |
| 2-hydroxyhexanal | — | — | — | — | 0.1 | — |
| 2-hydroxyoctanal | — | — | — | — | — | 0.2 |
| Amodimethicone | 0.8 | 0.8 | 0.8 | 0.8 | 0.8 | 0.8 |
| Water, preservative, | up to | up to | up to | up to | up to | up to |
| perfume oils | 100 | 100 | 100 | 100 | 100 | 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent comprising, based on its weight,
a) from about 0.001 to about 10 wt. % of at least one succinimidyl ester, and
b) from about 0.001 to about 20 wt. % of at least one proteolipid of formula (II)
R′—X—R″ (II),
in which
R′ represents a straight-chain or branched, saturated or unsaturated hydrocarbon functional group having 11 to 24 carbon atoms,
R″ represents a protein, a peptide or a protein hydrolyzate,
X represents —C(O)O— or —N⁺(R^{III}₂)—R^{IV}— or —N(R^{III})R^{IV}— or —C(O)—N(R^{V})R^{VI},
R^{III} represents —(CH₂)ₓ—CH₃, where x=0-22, and
R^{IV} represents —CH₂—CH(OH)—CH₂— or —(CH₂)_{X}—, where x=0-22; and
R^{V} and R^{VI} represent, independently of one another, —H or —(CH₂)_{X}—CH₃, where x=0-22;
wherein R″ represents keratin or a keratin hydrolyzate when X represents —C(O)O—.

2. The hair treatment agent according to claim 1, characterized in that the hair treatment agent comprises, based on its weight, from about 0.01 to about 10 wt. %, succinimidyl ester(s) of formula (I) 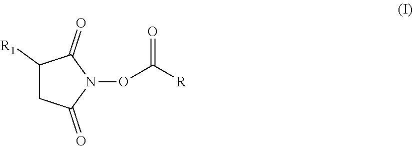
in which
R1 represents —H or an ionic group, and
R represents an optionally substituted saturated or unsaturated, linear, branched or cyclic, aliphatic or aromatic hydrocarbon functional group having at least 5 C atoms.

3. The hair treatment agent according to claim 1, characterized in that the hair treatment agent comprises, based on its weight, from about 0.01 to about 10 wt. %, of at least one succinimidyl ester from the group 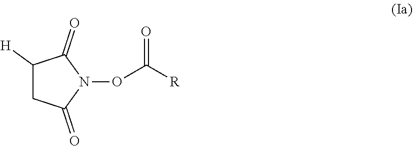
in which R represents -Ph or —(CH₂)nCH₃, where n=4, 5, 6, 7, 8, 9 or 10, 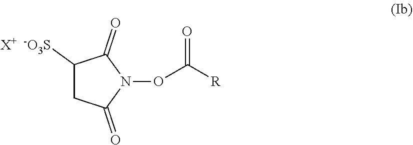
in which R represents -Ph or —(CH₂)nCH₃, where n=4, 5, 6, 7, 8, 9 or 10, and X represents H, a monovalent cation, or the n-th part of an n-valent cation,

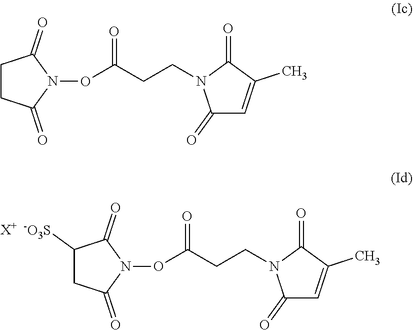

4. The hair treatment agent according to claim 1, characterized in that the hair treatment agent comprises, based on its weight, from about 0.01 to about 15 wt. %, of at least one proteolipid of formula (II), in which R″ represents keratin or a keratin hydrolyzate.

5. The hair treatment agent according to claim 1, characterized in that the hair treatment agent comprises, based on its weight, from about 0.01 to about 15 wt. %, of at least one proteolipid of formula (II), in which X represents —C(O)—O— or —N⁺(CH₃)₂—CH₂—CH(OH)—CH₂— and R′ represents —(CH₂)₁₁₋₂₃—CH₃, in particular —(CH₂)₁₇—CH₃.

6. The hair treatment agent according to claim 1, characterized in that the hair treatment agent comprises, based on its weight, from about 0.3 to about 20 wt. %, of amphoteric surfactant(s).

7. The hair treatment agent according to claim 1, characterized in that the hair treatment agent comprises, based on the weight of the agent, from about 0.01 to about 3 wt. %, of at least one polymer from the group of cationic cellulose polymers and/or cationic guar derivatives.

8. The hair treatment agent according to claim 1, characterized in that the hair treatment agent comprises, based on the weight of the agent, from about 0.05 to about 20 wt. %, behenyl trimethyl ammonium chloride.

9. The hair treatment agent according to claim 1, characterized in that the hair treatment agent additionally comprises, based on the weight of the agent, from about 0.001 to about 20 wt. % of at least one alpha-substituted aldehyde.

10. The hair treatment agent according to claim 1, characterized in that the hair treatment agent additionally comprises, based on the weight of the agent, from about 0.01 to about 10 wt. %, alpha-substituted aldehyde(s) of formula (III)
Y—CH(X)—CHO (III)
in which
X represents —OH, —Cl, —Br, —I, —O—(CH₂)ₙ, where n=1, 2, 3 or 4, or —O—(CH₂)ₘ—OH, where m=1, 2 or 3
Y represents —H, or —CH₃ or H₃C—(CH₂)ₖ—, where k=1, 2, 3, 4, 5, 6, 7, 8 or —OH or —(CH₂)ₚ—OH, where p=1, 2, 3, 4 or —CHO.

11. The hair treatment agent according to claim 1, characterized in that the hair treatment agent comprises, based on its weight, from about 0.01 to about 10 wt. %, of at least one alpha-substituted aldehyde from the group
2-hydroxypropanal (X=—OH, Y=—CH₃): 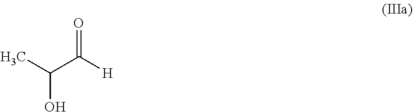
2-hydroxyhexanal (X=—OH, Y=H₃C—(CH₂)ₖ—, where k=3): 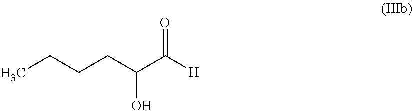
2-hydroxyoctanal (X=—OH, Y=H₃C—(CH₂)ₖ—, where k=5): 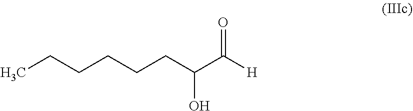
bromomalonaldehyde (X=Br, Y=—CH═): 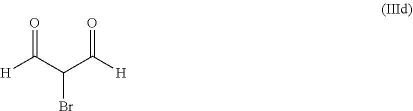
2-(2-hydroxyethoxy)acetaldehyde (X=—O—(CH₂)ₚ—OH, where p=2, Y=—H) 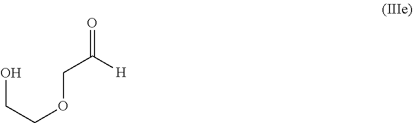
glyceraldehyde (X=—OH, Y=—(CH₂)ₚ—OH, where p=1).

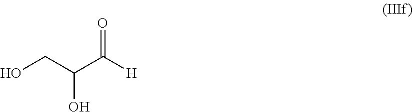

12. The hair treatment agent according to claim 1, characterized in that the hair treatment agent comprises, based on its weight, from about 0.001 to about 20 wt. % complexing agents from the group tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriacetic acid (HEDTA).

13. The hair treatment agent according to claim 1, characterized in that the hair treatment agent comprises, based on its weight, from about 0.001 to abut 20 wt. %, complexing agents from the group
a. tetrasodium-N,N-bis(carboxylatomethyl)-L-glutamate (tetrasodium glutamate diacetate, GLDA), 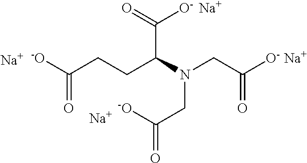
b. pentasodium diethylenetriaminepentaacetate (DTPA) 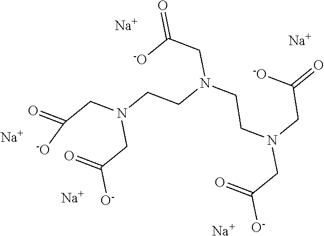
c. tetrasodium iminodisuccinate (IDS).

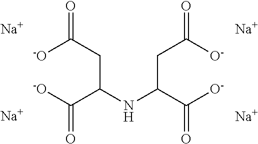

14. A method for treating hair comprising:
applying to wet or dry hair a hair treatment agent comprising, based on its weight,
c) from about 0.001 to about 10 wt. % of at least one succinimidyl ester, and
d) from about 0.001 to about 20 wt. % of at least one proteolipid of formula (II)
R′—X—R″ (II),
in which
R′ represents a straight-chain or branched, saturated or unsaturated hydrocarbon functional group having 11 to 24 carbon atoms,
R″ represents a protein, a peptide or a protein hydrolyzate,
X represents —C(O)O— or —N⁺(R^{III}₂)—R^{IV}— or —N(R^{III})R^{IV}— or —C(O)—N(R^{V})R^{VI},
R^{III} represents —(CH₂)ₓ—CH₃, where x=0-22, and
R^{IV} represents —CH₂—CH(OH)—CH₂— or —(CH₂)_{X}—, where x=0-22; and
R^{V} and R^{VI} represent, independently of one another, —H or —(CH₂)_{X}—CH₃, where x=0-22:
wherein R″ represents keratin or a keratin hydrolyzate when X represents —C(O)O—,
leaving the hair treatment agent on the hair for a selected period of time, and
subsequently rinsing out the hair treatment agent.

15. The method for treating hair of claim 14, wherein the hair treatment agent is left on the hair until the next time the hair is washed.

16. The method of claim 14, wherein the hair treatment agent is adapted to reduce the washout of color from chemically colored hair.

17. The method for treating hair of claim 14, wherein the selected period of time is from 10 to 300 seconds.
